(19) United States
(12) Patent Application Publication
(10) Pub. No.: US 2007/0225632 A1
(43) Pub. Date: Sep. 27, 2007

(54) HYDRATABLE POLYMERIC ESTER MATRIX FOR DRUG ELECTROTRANSPORT

(76) Inventors: David Rauser, Gilroy, CA (US); David E. Edgren, Los Altos, CA (US); Janardhanan A. Subramony, Santa Clara, CA (US); Rama V. Padmanabhan, Los Altos, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/688,630

(22) Filed: Mar. 20, 2007

Related U.S. Application Data
(60) Provisional application No. 60/784,849, filed on Mar. 21, 2006.

(51) Int. Cl. A61N 1/30

(52) U.S. Cl. 604/20

(57) ABSTRACT

A transdermal electrotransport drug delivery system to an individual. The system has a liquid imbibing polymer with carboxyl groups available for noncovalently associating with a cationic drug. The liquid imbibing polymer is applicable for imbibing liquid before the device is deployed on a patient for electrotransport drug delivery.

### CROSS REFERENCE TO RELATED U.S. APPLICATION DATA

The present application is derived from and claims priority to provisional application U.S. Ser. No. 60/784,849, filed Mar. 21, 2006, which is herein incorporated by reference in its entirety.

### TECHNICAL FIELD

This invention relates to a medical device for transdermal administration of a drug and to a method of treating a subject by administering a drug to a patient with the medical device. In particular, the invention relates to transdermal electrotransport systems for administration of a drug with a hydratable drug reservoir.

### BACKGROUND

In an animal, the natural barrier function of the body surface, such as skin, presents a challenge to delivery of therapeutics into circulation. Transdermal devices for the delivery of biologically active agents or drugs have been used for maintaining health and therapeutically treating a wide variety of ailments. For example, analgesics, steroids, etc., have been delivered with such devices. Transdermal drug delivery can generally be considered to belong to one of two groups: transport by a “passive” mechanism or by an “active” transport mechanism. In the former embodiment, such as drug delivery skin patches, the drug is incorporated in a solid matrix, a reservoir, and/or an adhesive system.

Most passive transdermal delivery systems are not capable of delivering drugs under a specific profile, such as by ‘on-off’ mode, pulsatile mode, etc. Consequently, a number of alternatives have been proposed where various forms of energy drive the flux of the drug(s). Some examples include the use of iontophoresis, ultrasound, electroporation, heat and microneedles. These are considered to be “active” delivery systems. Iontophoresis, for example, is an “active” delivery technique that transports solubilized drugs across the skin by an electrical current. The feasibility of this mechanism is constrained by the solubility, diffusion and stability of the drugs, as well as electrochemistry in the device.

A significant advantage of active transdermal technologies is that the timing and profile of drug delivery can be controlled, so that doses may be automatically controlled on a pre-determined schedule or self-delivered by the patient based on need. For example, U.S. Pat. Nos. 5,057,072; 5,084,008; 5,147,297; 6,039,977; 6,049,733; 6,181,963, 6,216,033, 6,317,629, and US Patent Publication 20030191946, are related to electrotransport transdermal delivery of drugs.

In iontophoretic systems, one electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the ionic substance to be driven into the body is positively charged, then the positive electrode (the anode) will be the active (or donor) electrode and the negative electrode (the cathode) will serve as the counter electrode. If the ionic substance to be delivered is negatively charged, then the cathodic electrode will be the active (or donor) electrode and the anodic electrode will be the counter electrode. Electrotransport devices require a reservoir or source of the active agent that is to be delivered or introduced into the body. Such reservoirs are connected to the anode or the cathode of the electrotransport device to provide a fixed or renewable source of one or more desired active agents.

Although electrotransport is useful for delivery of ionic drugs, not all ionic drugs are suitable for such delivery. Drug stability, both in use and during storage, is important for the manufacture and of pharmaceutical products. It is important to find a formulation that will provide acceptable stability for the active pharmaceutical ingredient for a period of storage, such as the recommended period before the expiration of which the drug should be used (shelf life). A drug cannot be incorporated into a product if the molecule is not stable in formulation. Thus, many drugs, although therapeutically useful and feasible to be delivered transdermally, would not be available to patients without ways to maintain the stability over a period time adequate for commercial channels of distribution and use.

Yet another challenge to achieve practical electrotransport delivery involves maintaining physical compatibility of the moisture-sensitive electrical components present within the delivery system with aqueous-based formulations in close proximity. Metallic components of the sensitive electrical circuitry, for example, can be subject to breakdown by corrosion if exposed to humidity or bulk water of aqueous-based formulations. Keeping the formulation in the dry state until just prior to use would promote stability of the dosage form during storage.

Drug reservoirs used in iontophoresis are typically aqueous based systems using hydrophilic polymers. This allows for maximum ion mobility and conductivity under the influence of an electric field. There are a large variety of drug reservoirs in the literature to date such as polyvinyl alcohol (PVOH) as well as cellulose based polymers. Most reservoirs contain drug salt dissolved in a solution. This form offers the simplest means of drug loading. In prior methods described for forming reservoirs, the problem of aqueous stability is not adequately addressed.

Attempts to solve the lack of aqueous stability of drugs within reservoirs include the use of hydratable systems. Hydration refers to the absorption of any solvent or agent so as to dissolve drug molecules and maintain them in ionic form for electrotransport application. Examples of systems that have been developed in which the drug-containing reservoir is hydrated prior to use are polyurethane based systems. Examples of prior disclosures on hydration of reservoirs include, for example, U.S. Pat. Nos. 5,236,412; 5,288,289; 5,533,972; 5,582,587; 5,645,527; 6,275,728; and 6,317,629, the disclosure of which are incorporated by reference in their entireties. However, slow hydration kinetics and long solvation times are some of the problems associated with hydratable systems. Thus, further improvements are needed for better systems for hydratable iontophoretic drug delivery system.

Although the transdermal delivery of therapeutic agents has been the subject of intense research and development for over 30 years, because of the above reasons thus far only a few drug molecules have been found to be suitable for transdermal electrotransport application. The present invention provides methodology and composition in which drugs can be incorporated into a reservoir while providing improved stability for electrotransport delivery.

### SUMMARY

This invention provides methodology and composition for improving loading of cationic drugs in an iontophoretic drug delivery system. In one aspect, a liquid imbibing polymer is provided that has carboxyl groups free for noncovalently associating with cationic drug or drugs. In another aspect, in the novel polymer of the present invention, the cationic drug can remain in dry form (e.g., dehydrated form) to maintain stability until the time of use, whereupon the drug reservoir can be hydrated via imbibition of a solution. Keeping the drug in dry form helps to improve the stability of the drug in the electrophoretic device. The drug-loaded polymer of the present invention has been shown to preserve the stability of hydrolytically labile cationic drugs. Liquid imbibition (e.g., hydration) of the loaded polymer with a suitable agent prior to use allows delivery of therapeutic drugs under electrotransport conditions.

In one aspect, the present invention provides a method of preparing an electrotransport device for drug delivery, including forming a hydratable reservoir matrix in the device and imbibing liquid in the matrix prior to deployment wherein the hydratable reservoir matrix already contains a cationic drug. The drug in the hydratable reservoir matrix is noncovalently associated with a liquid imbibing polymer. As used herein, the term “matrix” refers to the structural or carrier material in the drug reservoir.

This invention introduces a new polymeric system for electrotransport drug delivery in which the drug-containing reservoir stabilizes compounds having poor solution stability in aqueous or organic solvents. The reservoir is infused with liquid (providing liquid to allow imbibition), which may swell the reservoir, prior to iontophoretic use where the onset of optimal delivery conditions is fast. However, if the matrix is made to have channels, no significant amount of swelling may be seen to occur during hydration. Furthermore, the method of loading drug ions onto polymers for improved stability and the synthesis of the polymeric ester based reservoir having free carboxyl groups for associating with a cationic drug through a condensation reaction are new to electrotransport applications.

The drug is loaded onto the polymer and preferably stored in an environment substantially free of aqueous or organic solvents. This method reduces or eliminates the major degradation pathways most common to the poor stability of many drug molecules.

The new polymeric material can act as an excellent reservoir material for electrotransport applications. Further, the reservoir according to the present invention hydrates rapidly prior to electrotransport activation.

In order to load and store therapeutically active drugs in this manner, the present invention provides a new liquid-imbibing polymeric ester that contains both free carboxylic acid groups and esterified carboxyl groups. Cationic drugs can be selectively loaded onto the carboxylic acid sites of the polymer by replacing the proton from the carboxyl group with a cationic drug in a concentrated solution of the drug. An effective loading solution dissociates the drug into ions and the cationic drug can replace the protons from the carboxyl groups of the polymer. The relative amount of drug ions loaded onto the polymer (which can be made into a film) with respect to the total amount of available carboxyl sites can also be controlled.

Conductivity values of prior dry drug containing reservoirs are often poor. One aspect of these polymeric films of the present invention is the quick absorption of water and applicable polar organic liquids. Once the polymer is hydrated, its conductivity is greatly increased. Fast hydration leads to shorter times to achieve usable conductive drug reservoirs.

### BRIEF DESCRIPTION OF THE DRAWINGS

The present invention is illustrated by way of example in embodiments and not limitation in the figures of the accompanying drawings in which like references indicate similar elements. The figures are not shown to scale unless indicated otherwise in the content.

FIG. 1 illustrates a schematic, exploded view of a typical electrotransport device in which reservoirs of the present invention can be used.

FIG. 2 illustrates a graph of Apomorphine flux (free base equivalent) from hydroxyethyl cellulose (HEC)-polyacrylic acid (CARBOPOL) films of the present invention treated with an aqueous solution containing antioxidants under a current density of 100 μA/cm².

FIG. 3 illustrates a graph showing a comparison of stability at 25° C. of Apomorphine in HEC-CARBOPOL dry films of the present invention and in aqueous solution.

FIG. 4 illustrates a graph showing a comparison of stability at 40° C. of Apomorphine in HEC-CARBOPOL dry films of the present invention and in aqueous solution.

FIG. 5 shows the structure of NATROSOL 250 hydroxyethyl cellulose.

FIG. 6 shows the structure of ethyl hydroxyethyl cellulose.

FIG. 7 shows the structure of a polyvinyl alcohol-polyethylene glycol graft copolymer.

FIG. 8 shows the infrared scan of an ester polymer of the present invention and scans of the two constituent polymers that formed the ester.

### DETAILED DESCRIPTION

The present invention relates to hydratable (liquid imbibing) ester polymer with both free and esterified carboxylic acid groups for transdermal delivery, especially such delivery by electrotransport (such as iontophoretic delivery on a body surface). Cationic drugs can be selectively loaded onto the carboxylic acid sites of the polymer by replacing the proton from the carboxyl group with a cationic drug.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain, but not agents (such as permeation enhancers) the primary effect of which is to aid in the delivery of another biologically active agent such as the therapeutic agent transdermally.

“Electrotransport” or “iontophoresis” refers to the delivery of pharmaceutically active agents (charged, uncharged, or mixtures thereof) through a body surface (such as skin, mucous membrane, eye, or nail) wherein the delivery is at least partially induced or aided by the application of an electric potential. The agent may be delivered by electromigration, electroporation, electroosmosis or any combination thereof. Electromigration involves the electrically induced transport of charged ions through a body surface by moving ions by means of a difference in electrical potential.

As used herein, the term “matrix” refers to a solid, or semi-solid substance, such as, for example, a polymeric material or a gel, that has spaces for a beneficial agent to populate and can hold a liquid for electrotransport. The matrix serves as a repository in which the beneficial agent is contained and may be porous.

As used herein, the term “therapeutically effective” refers to the amount of drug or the rate of drug administration needed to produce the desired therapeutic result.

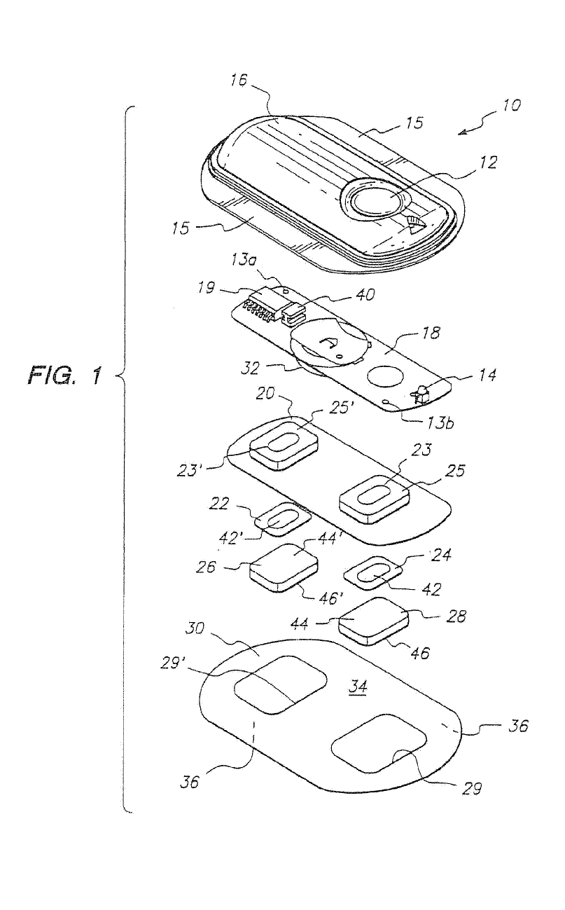

The ester polymer of the present invention can be used in electrotransport systems, such as many of the prior disclosed electrotransport systems. For example, electrotransport systems such as those of U.S. Pat. Nos. 6,181,963; 6,317,629; and others can incorporate reservoirs having the ester polymer drug matrix of the present invention. An iontophoretic system similar to that of U.S. Pat. No. 6,181,963 is shown in FIG. 1. FIG. 1 shows a perspective exploded view of an electrotransport device 10 having an activation switch in the form of a push button switch 12 and a display in the form of a light emitting diode (LED) 14. Device 10 includes an upper housing 16, a circuit board assembly 18, a lower housing 20, anodic electrode 22, cathodic electrode 24, anodic reservoir 26, cathodic reservoir 28 and skin-compatible adhesive 30. Upper housing 16 has lateral wings 15 that assist in holding device 10 on a patient's skin. Upper housing 16 is preferably composed of an injection moldable elastomer (e.g. ethylene vinyl acetate).

Printed circuit board assembly 18 includes an integrated circuit 19 coupled to discrete electrical components 40 and battery 32. Printed circuit board assembly 18 is attached to housing 16 by posts (not shown) passing through openings 13a and 13b, the ends of the posts being heated/melted in order to heat weld the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of printed circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′, 44 of reservoirs 26 and 28. The bottom sides 46′, 46 of reservoirs 26,28 contact the patient's skin through the openings 29′,29 in adhesive 30. Such a device can include a matrix of the ester polymer of the present invention in the system.

A reservoir, e.g., a cationic drug donor reservoir, contains the polymeric ester of the present invention. The polymeric ester is a polymer having a monomer component that is an acid polymer and a monomer component that is a hydroxyl polymer. The ester is prepared by a condensation reaction between the free carboxyl groups of an acid polymer with the hydroxyl groups of a second polymer (an hydroxyl polymer) to form a covalent ester cross-link. It is preferred that the hydroxyl polymer have multiple hydroxyl groups and the acid polymer have multiple carboxyl groups for cross-linking. A class of substance useful as the hydroxyl polymer is hydroxyalkyl polymer. Such a hydroxyalkyl polymer will have hydroxyl group —OH connected to another group through an alkyl linkage in the polymer, i.e., having a —OH connected via single bonded hydrocarbon link (e.g., —CH₂—) to other groups in the polymer. Preferably, the —OH is connected via a single bonded hydrocarbon link to an oxygen in an ether linkage. Preferably, the single bonded hydrocarbon link is one to three carbons long. More preferably the single bonded hydrocarbon link is one to two carbons long, e.g., —CH₂—CH₂— as in a hydroxyethyl group. Further, it is preferred that there are ether linkages connecting repeated moieties in the polymer, as in for example, polyethylene glycol polymer, alkylene oxide (e.g., ethylene oxide, propylene oxide)polymer, and carbohydrate like structures.

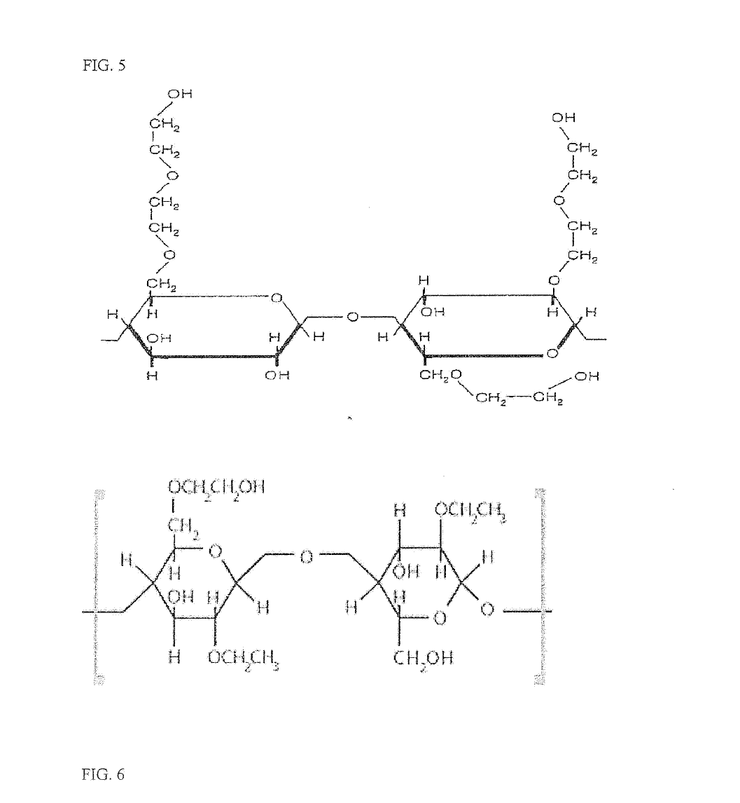

A useful type of hydroxyalkyl polymer includes carbohydrates such as polysaccharides and their derivatives. Such carbohydrates and their derivatives contain polymerized saccharose ring structures. Carbohydrate derivatives are useful as long as they have hydroxyl groups, especially primary or secondary hydroxyl group, that can form ester with an acid polymer. Preferably the hydroxyl polymer is cellulosic as a cellulose derivative. Preferred cellulosic hydroxyl polymers include hydroxyalkyl cellulose such as hydroxyethyl cellulose, hydroxypropyl cellulose, hydroxypropyl methyl cellulose, ethyl hydroxyethyl cellulose, and the like. FIG. 5 shows the structure of NATROSOL® 250 hydroxyethyl cellulose (presently at 2006 A.D. available from Hercules Inc., Wilmington, Del. 19894 U.S.A.). FIG. 6 shows the structure of an ethyl hydroxyethyl cellulose. One of the advantages afforded by the polysaccharides and especially cellulosic hydroxyl polymers is their liquid absorbing capacity, particularly in absorbing aqueous solutions. Another advantage is that they can form films with good mechanical properties such as flexibility and toughness. Other preferred hydroxyl polymers include starch and starch derivatives, maltodextrin, chitosan, and natural gums such as locust bean gum, guar gum, carrageenin, agar, and carob gum, and their derivatives.

Another class of hydroxyl polymers is linear polymers without ring structures, preferably with hydroxyl groups at both ends of the polymer. For example, hydroxyl polymers with blocks of ethylene oxide units are useful. Examples of such ethylene oxide containing hydroxyl polymers include polyvinyl alcohol-polyethylene glycol graft copolymer and ethylene oxide-propylene oxide-ethylene oxide triblock copolymers.

Polyvinyl alcohol-polyethylene glycol graft copolymer is also a preferred hydroxyl polymer for forming the ester. The polyethylene glycol chains of this polymer have primary —OHs at the ends thus providing the needed reactivity and additionally the graft copolymer inherently has good film forming and tensile properties. FIG. 7 shows the structure of a polyvinyl alcohol-polyethylene glycol graft copolymer.

Among the hydroxyl polymers, the ones preferred are those with a reactive —OH group in the primary hydroxyl position (i.e., the carbon bonded with the —OH group is connected via only single bonds to hydrogen atoms and only one carbon atom). Secondary hydroxyl groups are those where hydrogen and two carbon atoms are single-bonded to the carbon atom that is covalently bonded to the —OH. Tertiary hydroxyl groups are those where three carbon atoms are single-bonded to the carbon atom that is covalently bonded to the —OH. The primary position allows the —OH to be more approachable at molecular scale during chemical reaction and therefore to be more reactive than —OH in the secondary or tertiary positions.

The acid polymer for forming the ester is a polymer having repeating units with acidic carboxyl groups such that when these carboxyl groups form a covalent bond and cross-link with the hydroxyl polymer, they result in a cross-linked ester and thus achieve a liquid-imbibing yet insoluble structure. Under appropriate condition of liquid incorporation, the matrix can have a gel-like consistency with homogeneous physical property throughout the matrix. Examples of such acid polymers include polyacrylic acid, polymethacrylic acid, polyethylacrylic acid, copolymers of methyacrylic acids such as ethyl acrylate/methacrylic acid copolymers, cellulose acetate phthalate, hydroxypropyl methylcellulose acetate succinate, hydroxypropyl methylcellulose phthalate, polyvinyl acetate phthalate, and cellulose acetate trimellitate, alginic acid, and pectic acid, gelatin, casein, arachin, glycinin, and zein, some of which are polypeptides and proteins. Such acid polymers can have pendant groups substituted and can be homopolymers or copolymers, as long as they have multiple carboxyl groups reactive to —OH groups in the hydroxyl polymer to form an ester.

To react with the hydroxyl polymer, especially preferred is polyacrylic acid. The polyacrylic acid can either be cross-linked or noncross-linked. However, if the polyacrylic acid is cross-linked, the amount of cross-linking is sufficiently low that the polyacrylic acid can absorb a large amount of water. Useful polyacrylic acids commercially available include CARBOPOL® polyacrylic acids (which are presently at 2006 A.D. available from Noveon, Inc., 9911 Brecksville Road, Cleveland, Ohio), such as CARBOPOL 907 (which is not cross-linked), CARBOPOL 980 (which is cross-linked), CARBOPOL 940 and CARBOPOL 2984, and the like. The more preferred polyacrylic acids are either soluble in water or can absorb a large amount of water (e.g., 100 times by weight, preferably more than 500 times by weight, more preferably more than 1000 times by weight) at about neutral pH to form a homogenous material. The viscosity of preferred polyacrylic acid when dissolved at a concentration of 0.5 weight percent in pH 7.5 buffer is preferably in the range of about 1,000 to 80,000 centipoises, preferably 40,000 to 60,000 centipoises as measured by a Brookfield viscometer at 20 revolutions per minute.

The reaction below illustrates an esterification reaction of the present invention, using the embodiment of hydroxyethyl cellulose (HEC) and polyacrylic acid (PAA) as an example.

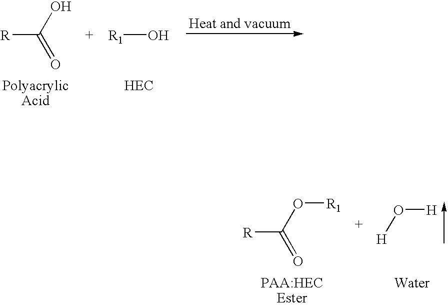

In the above reaction, the polyacrylic acid is formed from polymerizing acrylic acid monomers to form homopolymer or copolymerizing acrylic acid with comonomer (such as alkyl acrylate and methylacrylic acid) to form copolymer. The polyacrylic acid can be cross-linked or noncross-linked. Cross-linking can be done, for example, with divinyl glycol, allylpentaerythritol, and the like, as known in the art. Thus, R is a group that contains a hydrocarbon (preferably all single-bonded) carbon chain backbone with pendant —COOH groups. Other than —COOH and H, preferably the pendant groups on the carbon chain backbone are alkyl group and acrylate group. In the case of cross-linked polyacrylic acid, there can be only a single such carbon chain cross-linked to itself or many chains cross-linked in R. For cross-linked polyacrylic acid, preferably, the molecular weight is such that if the cross-linked polyacrylic acid were without cross-linker (i.e., made from the same ingredients but without using cross-linker), the weight average molecular weights are about 200,000 to 1,000,000, preferably 400,000 to 600,000 as measured by gel permeation chromatography using linear polyacrylic acid as reference. Therefore, in the polyacrylic acid, there are many —COOH groups that can react with the hydroxyl polymer. It is noted that other acid polymers disclosed above, e.g., polymethacrylic acid, polyethylacrylic acid, etc., may be used to form ester with a hydroxyl polymer in a similar fashion.

In hydroxyethyl cellulose, R1 contains a polysaccharide group of the hydroxyl polymer. When other hydroxyl polymers are used, R1 can take many forms, as long as the —OH attached to it is reactive with the polyacrylic acid. The condensation reaction of the carboxyl group and the hydroxyl group of the reacting polymers uses the carboxyl groups to covalently bond with the hydroxyl group through the loss of a water molecule, thereby resulting in a cross-link in the ester bond. An energy source is used to drive the reaction. The currently preferred energy source is thermal energy combined with vacuum. Other energy sources include electromagnetic radiation such a microwave or irradiation with radioactive source.

When not all of the carboxyl groups in the acid polymer react in the esterification, the unesterified —COOH groups in the ester polymer under the right pH condition in a polar solvent will allow dissociation of the hydrogen ion and result in —COO⁻, which can noncovalently, but rather ionically, associate with a cationic drug. Such —COO ionized functional groups are immobile in the ester polymer matrix. Further, because the —COO⁻ ionized functional groups are part of the integral molecular structure of the ester polymer, such —COO⁻ ionized functional groups are different from ion exchange materials that are incorporated as beads, particles, or other nonhomogeneous phase separated mixtures in a gel, such as those in a reservoir with ion exchangers dispersed in a gel. Such ion exchangers are not covalently linked to the rest of the matrix and lack hydration capacity. In the present system, the gel is preferably homogenous or substantially homogeneous.

The amount of drug loaded onto the polymer drug reservoir is dependent on the number of unreacted carboxylic acid sites of the ester polymer. The present invention takes advantage of unesterified carboxyl groups that are not involved in the reaction in the resulting cross-linked polymer. For example, in the above reaction, after the reaction, some of the —COOH groups in R may remain unreacted with the hydroxyl groups of the hydroxyl polymer. Similar results of having unreacted carboxyl groups left over can be achieved with other acid polymers and hydroxyl polymers. The amount of carboxylic acid sites for drug loading can be controlled by the stoichiometric amounts of acid polymer and hydroxyl polymer, such as HEC and polyacrylic acid (e.g., CARBOPOL, available from Noveon, Inc., 9911 Brecksville Road, Cleveland, Ohio) added during manufacturing.

However, because not all —OH groups in the hydroxyl polymer are equally reactive, it is possible to provide the hydroxyl polymer with more available —OH groups than the —COOH groups in the acid polymer. The primary —OH will be more reactive and thus the secondary or tertiary —OH would be less likely to participate in the esterification reaction. Further, even the primary —OH may not all form cross-links with the —COOH. On the other hand, even with the excess of —OH groups, not all the —COOH would react under normal atmospheric condition or under vacuum of 600 to 760 mmHg negative pressure compared to atmospheric pressure. Thus, there would be —COOH groups even with excess —OH groups in the hydroxyl polymer in the reacting mixture. To control the characteristics of the ester polymer the amount of available carboxyl groups to be loaded can be controlled. Methods include altering the total amount of polymer as well as changing the concentration and ratios of the acid polymer and hydroxyl polymer reactants used in synthesizing the polymer ester.

In the acid polymer and hydroxyl polymer before esterfication, the —OH/—COOH ratio generally ranges from about 1 to 10, preferably from about 2 to 5. The exact ratio may vary depending on the particular acid polymer and hydroxyl polymer selected for the reaction. For example, for HEC/PAA films (e.g., NATROSOL® 250/CARBOPOL 980) the —OH/—COOH ratio can be about 2 to 4.5 with a preferred range of about 2.5 to 4.

The ratios of hydroxyl polymer to carboxyl polymer can be determined experimentally to identify practical ranges. In general, using a lower amount of acid polymer (e.g., using a lower concentration of polyacrylic acid) will yield an ester polymer film that when hydrated is jelly-like with low mechanical integrity. Generally, to form reservoirs for iontophoretic drug delivery, the ester polymer in film form is a convenient structure. Such a film can be cut into small sizes to be placed in an iontophoretic device. A larger amount of the acid polymer in the reaction (e.g., using a higher concentration of PAA) would result in an ester polymer film that in the dry state is too brittle to handle. For example, using the same wt % solutions of PAA and HEC, with PAA solution ranging about 10 to 30 vol % in the mixture is suitable, with about 15 to 25 vol % being preferred, to avoid extremes in mechanical properties. In view of the present disclosure, one skilled in the art will know other variations of wt % solutions of each reactant and the mixture vol % to use for the two solutions. Although it is possible to use a mixture of polymers and a mixture of hydroxyl polymers, it is preferred that the esterification is between only one type of acid polymer and one type of hydroxyl polymer.

Synthesis of the polymeric ester can be done through a condensation reaction potentiated by heat and vacuum between the free carboxyl groups of the carboxyl polymers and the free hydroxyl of hydroxyl polymers to form a covalent ester cross-link. The cross-link causes the resulting polymeric ester to become insoluble in water (thereby permitting less polymer residue being left on the body surface, e.g., skin, when the delivery system is removed therefrom).

The following is a description of an embodiment of making the ester polymer. To prepare the ester, generally, a dilute aqueous solution of the hydroxyl polymer (e.g., hydroxyethyl cellulose) and an aqueous solution of the acid polymer (e.g., CARBOPOL polyacrylic acid) are prepared and mixed together. (Some polyacrylic acids, although slightly cross-linked, can still swell in aqueous liquid without particulates present and have the appearance of a liquid.) Concentration ranges for the solutions are preferably 1-10 wt % with a more preferred range being 2-5 wt % for ease of mixing and reacting. The two solutions of hydroxyl and acid carboxyl polymers are mixed at ratios of 95:5 to 60:40 with the preferred range being 85:15 to 75:25. The esterification is effected by heating at a temperature below the boiling point of the mixture solution.

Preferably before the condensation reaction the mixed solutions are heated for pre-drying the copolymer solution, preferably at a temperature range of 30-60° C., more preferably with a temperature range of 40-50° C. for a period of time (e.g., 12-48 hours). The mixture can be dried to a consistency of about a sticky liquid. This pre-dry heating removes most of the solvent water prior to the condensation reaction that releases water of the reaction. Cross-linking to form the ester linkages between the hydroxyl polymer and the acid polymer (e.g., HEC and CARBOPOL polyacrylic acid) is preferably done by vacuum curing in a vacuum oven (generally at a vacuum of about more than 700 mm of Hg of negative pressure compared to atmospheric pressure) at a temperature of, for example, in the range of about 40-80° C., with a preferred temperature of 50-55° C. for a period of time for removal of liquid to result in a dry ester polymer for further process and loading of drug. The vacuum drying period is typically 12-48 hrs. This process is applicable for HEC/CARBOPOL as well as for esterification of other acid polymers and carboxyl polymers. After esterification, the resulting ester polymer has esterified carboxyl groups and unesterified carboxyl groups in the cross-linked structure.

The dry ester polymer typically has an equilibrium moisture content at 50% relative humidity of about 3 wt % to about 10 wt %. What the exact content of water at this point is not critical so long as it can be cut into units (e.g., pieces of squares or disks) of suitable size and shape for further processing to load a desirable drug and implementation in an iontophoretic drug delivery device. If need be, the ester polymer can be placed temporarily into a humid environment of 75% to 95% to impart flexibility and fracture resistance to allow further processing such as die cutting or stamping. Preferably, the ester polymer is dried into a layer of 0.5 mm to 3 mm for ease of liquid absorption later. The dry ester polymer can be cut into the size and shape desirable for further processing, e.g., 0.1 to 30 cm. Of course, depending on the specific electrotransport device in which the ester polymer unit is to be used, the size and shape of ester polymer unit can vary by a person skilled in the art. Final polymer geometry can be of most shape, size, and thickness.

A cationic drug (or drugs) can be loaded onto the acid polymer/hydroxyl polymer copolymer unit by means of imbibition of the drug in solution form. A specific drug may have a particular liquid that is more suitable as a solvent for the drug. For example, units of the copolymer can be placed in the drug solution and shaken in a shaker for a period of time for the drug to equilibrate in the copolymer matrix structure. Drug loading is governed by factors such as pH and type of solvent used for the loading solution and temperature. For example, for a cationic drug, a higher relative drug loading can be achieved in aqueous concentrated solutions of drug at higher pH than identical solutions of the same drug concentration at lower pH. The pH of the drug loading solution (i.e., the drug solution for loading the drug in the matrix in the drug loading process prior to hydration) is a factor that determines the amount of deprotonated acids sites for drug binding. At very high pH, the carboxylic acid group will be deprotonated and all groups will therefore be available for drug binding/loading. At very low pH, no drug loading or binding is possible because all acid groups will be protonated. The deprotonated acid groups are available for drug loading or binding to cationic drugs. Since the acid polymer has a pKa (acid dissociation constant) the pH of the drug loading solution will determine the fraction of acid protonated and deprotonated.

Units (pieces) of the cross-linked polymer with imbibed drug solution can then be rinsed using a solvent to remove drug solutions from the surface of the units before drying. A higher vacuum will facilitate the speed of drying. Thus, a vacuum of more than 700 mm of Hg of negative pressure compared to atmospheric pressure is generally preferred. The drying temperature is typically 30° C. to 60° C., preferably 40° C. to 50° C. Typically, a lower drying temperature will effect less drug degradation. Drying time varies depending on the drug and solvent used, but generally ranges from 12 hours to 72 hours for aqueous drug solution. After drying, the drug content in the dry copolymer units can be assessed by various analytical methods, such as by measurement of weight gain. The moisture content of the dry ester polymer units at 50% relative humidity is typically about 3 wt % to 10 wt %, preferably about 4 wt % to 6 wt % for facilitating storage to maintain drug stability. In view of the present disclosure, such esterification reaction, drug imbibition and drying, as well as weight gain assessment techniques are within the knowledge of one skilled in the art.

Prior to eletrotransport, the dry drug-containing polymer must be treated with an ion freeing solvent, through which the ionic drug can move by the application of an electrical potential. Such a treatment is generally done with a liquid solvent or solution of with polar solvent and is called “hydration” herein. The hydration step allows the bound drug molecules to dissociate from the carboxyl groups and can be any aqueous or polar organic solvent that will allow the drug ions to flow under the influence of an electric field. The dry ester polymer units can be hydrated by liquid (or solvent) imbibition before drug delivery is commenced on a patient. Typically the ester polymer unit will swell as liquid is being imbibed. Hydrating the ester polymer with a solvent or solvent mixture requires the use of a polar liquid capable of solvating the drug ion and preserving it in an ionic state for electrotransport delivery. Solvents used for this include organic solvents, inorganic solvents, solution of various solvents, buffers, and the like that one skilled in the art will know related to the drug. Such solvents include, but not limited to: water, ethanol, ethanol: water blends (especially useful at 70:30 to 30:70 ratios), methanol, methanol:water blends, glycerin, glycerin: water blends, propylene glycol, propylene glycol: water blends, dimethyl sulfoxide, dimethyl sulfoxide: water blends, glycerol oleate solution, low molecular weight polyethylene glycol (PEG, e.g., PEG 400), PEG: water blends, PEG 660 12-hydroxy stearate (note: paste at room temp but liquid at skin temp), and combinations thereof

Although a wide range of the amount of liquid infusion can be used, the ester polymer matrix before hydration (either as a matrix before drug loading or as a dry matrix after drug loading) typically can be allowed to imbibe liquid in the amount of about 10 volume percent (vol %) to 75 vol %, preferably about 15 vol % to 50 vol %, more preferably about 15 vol % to 30 vol %. With liquid imbibition, the volume of the ester polymer matrix can increase about 10% to 75 vol %, preferably about 15 vol % to 50 vol %, more preferably about 15 vol % to 30 vol %. The hydratable polymer matrix can be allowed to imbibe liquid to result in wt % changes similar to vol % ranges above. After hydration, the ester polymer matrix may become a gel or gel-like substance. However, the gel or gel-like substance will not completely dissolve in the solvent due the presence of ester cross-links. The drug concentration after hydration is about 0.5 wt % to 20 wt %, preferably about 15 wt % to 10 wt % and is suitable for electrotransport delivery.

Hydration can be done using, for example, a pipette or syringe type of device or other devices that provide a controlled volume of hydrating liquid. Typically, the length of operation after the hydration step is short enough to preserve drug stability. However, additives to the hydrating agent such as antioxidants can be used to preserve drug stability when there is a need to protect against any short-term instability. Furthermore, the hydration media can be formulated to provide for ideal electrotransport conditions, such as operating pH, allowing they effectively free the cationic drug from the polymer host.

Various biologically active agents or drugs may be incorporated in the ester polymer matrix of the present invention for use in treating individual in need of treatment by such drugs. The biologically active agents or drugs can be incorporated by imbibition and drying. The drug containing matrix can then be hydrated before drug delivery. Such biologically active agents or drugs include cationic drugs that are known to those skilled in the art. Agent or drugs that can be incorporated into the ester polymer matrix include, for example, interferons, alfentanyl, amphotericin B, angiopeptin, baclofen, beclomethasone, betamethasone, bisphosphonates, bromocriptine, buserelin, buspirone, calcitonin, ciclopirox, olamine, copper, desmopressin, diltiazem, dobutamine, dopamine agonists, dopamine agonists, doxazosin, droperidol, enalapril, enalaprilat, fentanyl and its analogs (such as alfentanil, carfentanil, lofentanil, remifentanil, sufentanil, trefentanil), encainide, G-CSF, GM-CSF, M-CSF, GHRF, GHRH, gonadorelin, goserelin, granisetron, haloperidol, hydrocortisone, indomethacin, insulin, insulinotropin, interleukins, isosorbide dinitrate, leuprolide, LHRH, lidocaine, lisinopril, LMW heparin, melatonin, methotrexate, metoclopramide, miconazole, midazolam, nafarelin, nicardipine, NMDA antagonists, octrebtide, ondansetron, oxybutynin, PGE 1, piroxicam, pramipexole, prazosin, prednisolone, scopolamine, seglitide, sufentanil, terbutaline, testosterone, tetracaine, tropisetron, vapreotide, vasopressin, verapamil, warfarin, zacopride, zinc, and zotasetron, individually or in combination.

The ester polymer is useful for incorporating agents or drugs such as peptides, polypeptides and other macromolecules typically having a molecular weight of at least about 300 daltons, and typically a molecular weight in the range of about 300 to 40,000 daltons. Specific examples of peptides and proteins in this size range include, without limitation, LHRH, LHRH analogs such as buserelin, gonadorelin, nafarelin and leuprolide, GHRH, insulin, heparin, calcitonin, endorphin, TRH, NT-36 (chemical name: N=[[(s)-4-oxo-2-azetidinyl]carbonyl]-L-histidyl-L-prolinamide), liprecin, pituitary hormones (e.g., HGH, HMG, HCG, desmopressin acetate, etc.,), follicle luteoids, aANF, growth hormone releasing factor (GHRF), βMSH, TGF-β, somatostatin, atrial natriuretic peptide, bradykinin, somatotropin, platelet-derived growth factor, asparaginase, bleomycin sulfate, chymopapain, cholecystokinin, chorionic gonadotropin, corticotropin (ACTH), epidermal growth factor, erythropoietin, epoprostenol (platelet aggregation inhibitor), follicle stimulating hormone, glucagon, hirulogs, hyaluronidase, interferons, insulin-like growth factors, interleukins, menotropins (urofollitropin (FSH) and LH), oxytocin, streptokinase, tissue plasminogen activator, urokinase, vasopressin, ACTH analogs, ANP, ANP clearance inhibitors, angiotensin II antagonists, antidiuretic hormone agonists, antidiuretic hormone antagonists, bradykinin antagonists, CD4, ceredase, CSF's, enkephalins, FAB fragments, IgE peptide suppressors, IGF-1, neuropeptide Y, neurotrophic factors, opiate peptides, parathyroid hormone and agonists, parathyroid hormone antagonists, prostaglandin antagonists, pentigetide, protein C, protein S, ramoplanin, renin inhibitors, thymosin alpha-1, thrombolytics, TNF, vaccines, vasopressin antagonist analogs, alpha-1 anti-trypsin (recombinant).

Other drugs that can be incorporated in the ester polymer matrix include diphenylmethane derivatives with antihistaminic activity such as cyclizine, chlorcyclizine, bromodiphenhydramine, diphenylpyraline, diphenhydramine, chlorcyclizine, medrilamine, phenyltoloxamine clemastine; pyridine derivatives with antihistaminic activity such as chlorpheniramine, brompheniramine, pheniramine, mepyramine, tripelennamine, chloropyramine, thenyidiamine, methapyrilene; diphenylmethane derivatives with anticholinergic activity such as adiphenine, piperidolate, benztropine, orphenadrine, chlorphenoxamine, lachesine, poldine, pipenzolate, clidinium, benzilonium, ambutonium; anticholinergic agents such as oxybutynin, oxyphenonium, tricyclamol, dicyclomine, glycopyrronium, penthienate; antidepressant drugs such as fluoxetine, iprindole, imipramine, clomipramine, desipramine, trimipramine, amitriptylline, nortriptylline, noxiptiline, butriptiline, doxepin, dothiepin, iprindole, protryptiline, melitracene, dimetacrine, opipramol, paroxetine, sertraline, citalopram; tranquillizers such as promazine, chlorpromazine, chlorproethazine, methoxypromazine, methpromazine, promethazine, dimethothiazine, methiomeprazine, trimeprazine, methiotrimeprazine, diethazine, thioridazine, perazine, trifluoperazine, thioperazine, thiethylperazine, perphenazine, fluphenarine thiopropazate, thiothixene, chlorprothixene; antipsychotics such as pimozide, thiopropazate, flupenthixol, clopenthixol, trifluoperazine, olanzapine; anorexics such as fenfluramine and chlorphentermine; analgesics such as methadone and dextropropoxyphene; local anaesthetics such as tetracaine, stadacaine, cinchocaine, lidocaine; antihypertensives such as propranolol, oxprenolol, acebutolol, sotalol, metoprolol; antiarrhythmic and antianginals such as amiodarone, dilthiazem and verapamil; antiestrogen such as tamoxifen; and antiosteoporotic agents such as raloxifen. Cationic drugs that are mentioned in U.S. Pat. No. 6,181,963 can also be used and are incorporated by reference herein.

Certain agents or drugs, especially biologics, proteins, polypeptides, polynucleotides, and the like, may degrade in solution rapidly. Some may have less than 90% recovery at room temperature within one week, or even less. Some may be unstable to the extent that recovery from solution is 80% or less in 3 weeks, 2 weeks, or even 1 week. Such drugs will benefit from employing the ester of the present invention for dry storage before hydration.

The ester polymer of the present invention is particularly suitable for associating with cationic drugs that are less stable, thus helping to stabilize the drugs. Typically, iontophoretic devices from the time of manufacture to being used may be in storage (e.g., in a warehouse, in a pharmacy, hospital, a doctor's office, or other places of transit or storage) ranging from weeks to months. Such storage would typically be at room temperature (e.g., about 27° C.) and environment. Thus, stability of iontophoretic devices longer than such shelf lives is desirable. The ester polymer matrix of the present invention is advantageously used for drugs that generally would otherwise have a short shelf life in liquid form, before being deployed on a patient, such as less than 12 months, about 0.01 month to 6 months, and about 0.1 to 1 month. By using the ester polymer matrix of the present invention, it is contemplated that many drugs remain stable in the dry matrix for many months. As used herein, “having a shelf life” of a period means that the drug will be consistently recoverable to at least 90 wt % of the amount originally present for at least the period specified when in storage in ambient environment at room temperature.

In additional embodiments, the drug reservoir in an iontophoretic delivery device of the present invention may optionally contain additional components such as, additives, permeation enhancers, stabilizers, dyes, diluents, plasticizer, tackifying agent, pigments, carriers, inert fillers, antioxidants, excipients, gelling agents, anti-irritants, vasoconstrictors, buffering agent, and other materials as are generally known to the transdermal art, provided that such materials are present below saturation concentration in the reservoir. Such materials can be included by one skilled in the art.

The drug reservoir having the ester matrix of the present invention can be placed in an electrotransport device such as one shown in FIG. 1, prior or after hydration. When placed in the device, the drug reservoir will be in contact with current distribution parts such as silver or silver chloride electrodes and can contact body surface after hydration.

### EXAMPLES

Below are examples of specific embodiments for carrying out the present invention. The examples are offered for illustrative purposes only, and are not intended to limit the scope of the present invention in any way. In the following examples all percentages are by weight unless noted otherwise.

### Example 1

### Preparation of HEC-CARBOPOL Polymeric Ester

Solutions of hydroxyethyl cellulose (hydroxyl class) NATROSOL 250 and CARBOPOL polyacrylic acid (carboxyl class) CARBOPOL 980 are prepared and mixed together. Concentration ranges for the solutions are 1-10 wt % with a preferred range being 2-5 wt %. The two solutions of hydroxyl and carboxyl class polymers are mixed at ratios of 95:5 to 60:40 for HEC: CARBOPOL with the preferred range being 85:15 to 75:25. The mixed solutions are then placed in a forced air oven for pre-drying the solution of polymers at a temperature range of 30-60° C. with a preferred temperature range of 40-50° C. for 12-48 hours. Cross-linking between the HEC and CARBOPOL to form the ester linkages is done by vacuum curing in a vacuum oven with a vacuum of 600-760 mm Hg and a temperature in the range of 40-80° C., with a preferred temperature of 50-55° C., for 12-48 hrs.

### Example 2

### Studies using Apomorphine Hydrochloride Hemihydrate

Drug loading, electrotransport, and stability studies were carried out with apomorphine hydrochloride hemihydrate as the model compound. Apomorphine is a highly unstable compound in water due to oxidation of the catechol moiety. Aqueous and some organic solutions of apomorphine turn bluish green indicative of oxidation.

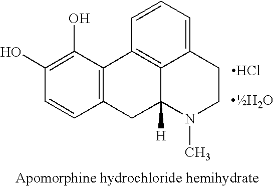

Solutions of HEC and CARBOPOL each at 1 wt % were prepared in separate glass jars using Milli-Q filtered water. The two solutions were mixed together at an 80:20 (1% HEC: 1% CARBOPOL) weight ratio and poured into plastic containers measuring approximately 300 mils (7.5 mm) in thickness. The container housing the solution was placed in a 50° C. oven until all solvent had been removed and a sticky film resulted. The polymer mixture was transferred to a vacuum oven (724 mm of Hg. vacuum) set at 80° C. for 24 hrs to perform the esterification reaction. Finally, the resulting copolymer films were punched into 0.38 in (9.5mm) diameter disks.

Apomorphine was loaded onto the HEC-CARBOPOL copolymer via a 50 mg/mL solution of the drug dissolved in methanol. The HEC-CARBOPOL disk was placed with 2 mL of the apomorphine solution and mixed on a shaker for 1 hour. The disks were then rinsed with methanol and dried in a vacuum oven 724 mm (28.5 in) of Hg vacuum at 30° C. for 16 hours. Apomorphine content in the films was assessed by weight. Table 1 shows the percent increase of film weight from the addition of apomorphine.

TABLE 1
Weight increase of HEC-CARBOPOL films from the addition of
apomorphine

| | Weight % |
|---|---|
| Film # | increase |
| 1 | 19.93 |
| 2 | 5.49 |
| 3 | 9.23 |
| 4 | 10.53 |
| 5 | 14.77 |
| Average | 11.99 |
| St Dev | 5.54 |

### Example 3

### In vitro Electrotransport Studies

Electrotransport experiments were performed using iontophoretic drug delivery systems that had a silver foil anode and a silver chloride cathode, similar to that shown in FIG. 1. The anodic compartment comprised a PVOH gel containing 80% sterile water and chloride ions (which acted as the chloride source) situated next to the silver foil and the HEC-CARBOPOL polymer loaded with apomorphine. The drug reservoir was separated from the chloride source by a Sybron anion exchange membrane. HEC-CARBOPOL polymers and films with drug were prepared as indicated above. To achieve and overall film thickness of 1/32 in (0.8 mm), multiple polymer disks were layered.

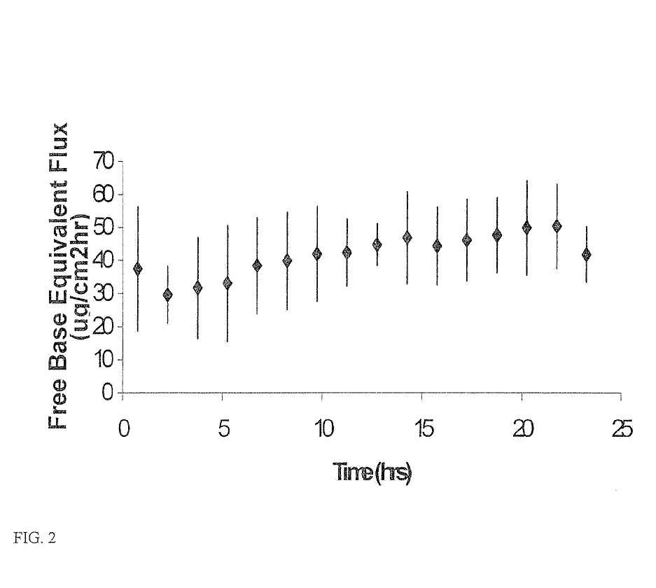

The polymer film layers were treated with an aqueous solution to dissolve the drug and provide ion mobility. The hydration solution contained 0.6 mg/mL citric acid, 0.3 mg/mL EDTA, and 0.06 mg/mL sodium metabisulfite used as antioxidants for apomorphine stability during use. The films were observed to swell upon treatment and subsequently re-punched to achieve the desired 0.38 in (9.5 mm) diameter suitable for a matrix in a drug reservoir prior to the start of the experiment. The cathode compartment had human heat separated skin contacting the HEC-CARBOPOL films containing a receptor solution of 10 mM citric acid with 15 mM NaCl (pH=5). The electrodes were connected to a DC power source that supplied a constant electric current of 0.100 mA/cm² (0.0712 mA). Receptor solution was analyzed by HPLC for drug content to provide a 24-hour delivery profile. FIG. 2 shows the apomorphine flux profile from HEC-CARBOPOL films treated with the aqueous solution containing antioxidants. Time was shown in hours and flux was shown in μg/cm² hr. In FIG. 2, the data points on the graph are average points and the vertical line through the data points show the standard deviations. The figure shows that an ester polymer matrix according to the present invention is applicable for iontophoretic drug delivery. It is contemplated that other cationic drugs can be delivered with a system with a reservoir with a matrix of the ester polymer.

### Example 4

### Stability Studies

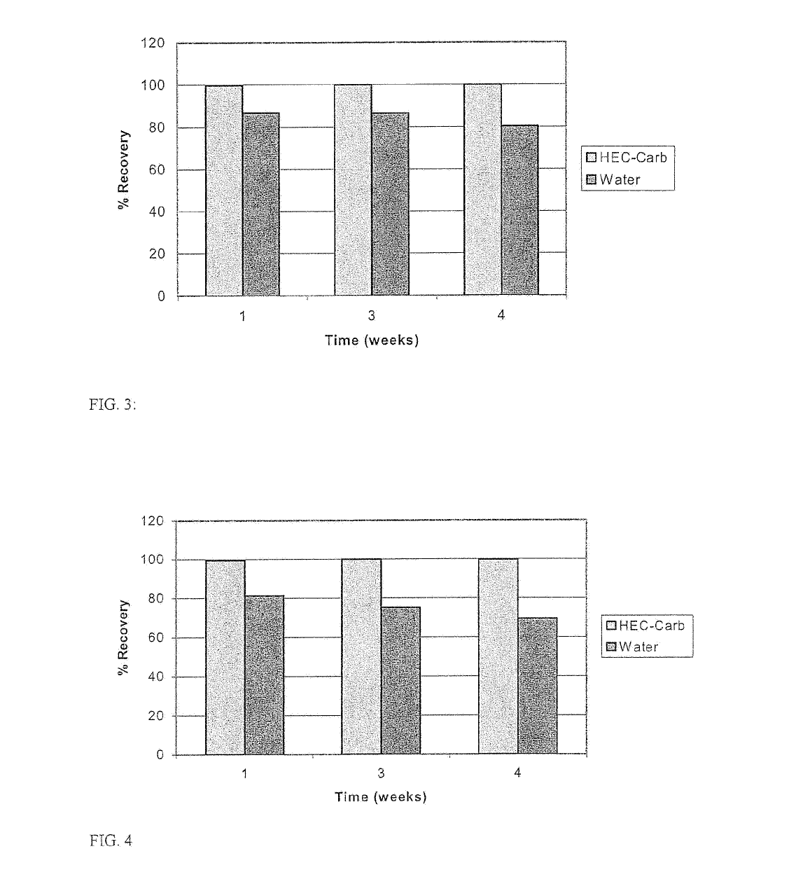

Apomorphine was loaded onto HEC-CARBOPOL polymers as described previously. The polymers were placed in plastic shells, stored in aluminum pouch stock and incubated at 25° and 40° C. A 100 ug/niL solution of apomorphine in Milli-Q filtered water was prepared to serve as a control. FIG. 3 and FIG. 4 show the results of the stability study, in percent recovery of drug originally present versus time in weeks. Apomorphine in the polymer was extracted in a solution containing 0.1% citric acid and 0.1% sodium metabisulfite and analyzed by HPLC. FIG. 3 shows the recovery of Apomorphine at 25° C in HEC-CARBOPOL films as compared to water. FIG. 4 shows the recovery of Apomorphine at 40° C. in HEC-CARBOPOL films as compared to water. Apomorphine loaded onto HEC-CARBOPOL films showed excellent stability at both temperatures throughout the 4 weeks study. Control groups show that apomorphine was highly unstable in water at both temperatures.

### Example 5

An ester copolymer of the present invention was formed between the acid polymer polyacrylic acid and a hydroxyl polymer containing ethylene oxide:propylene oxide:ethylene oxide triblock copolymer. First, polyacrylic acid was dissolved in water at a concentration of 0.40 g CARBOPOL/g water. The polyacrylic acid (PAA) was commercially available CARBOPOL 980 manufactured by Noveon, Incorporated, Cleveland, Ohio. The viscosity of this grade dissolved at a concentration of 0.5 weight percent in pH 7.5 buffer is specified to be in the range of 40,000 to 60,000 centipoises as measured by a Brookfield viscometer at 20 revolutions per minute. Next, 0.5084 grams of the triblock copolymer was added to the water/CARBOPOL mixture. The triblock copolymer was commercially available LUTROL® F68 (“F68”) manufactured by the BASF Corporation, Mount Olive, N.J. This a:b:a triblock copolymer has a molecular weight of approximately 7,680 to 9,510 grams per mole where the “a” represents approximately 80 ethylene oxide repeat units and “b” represents approximately 27 propylene oxide repeat units. This linear polymer has two terminal hydroxyl groups, one located at each end of the polymer chain. This mixture was transferred to a vacuum oven where it was treated for 11 days in vacuum at a temperature of 50° C. to produce the ester polymer of the present invention.

Two experiments were conducted to test the presence of ester cross-linking. First, a small sample of the resulting ester polymer was cut into small pieces and soaked in acetone to swell and soften them. Then, the samples were compressed with a rubber pressing block onto the surface of an Attenuated Total Reflectance Crystal accessory of a Nicolet Magna IR 760 Infrared Spectrometer. The acetone was dried off in a fume hood. The resulting sample was scanned 200 scans at frequency (i.e., 1/wavelength) range from 1,900 cm⁻¹ to 900 cm⁻¹ with a resolution of 4 cm⁻¹. Then, a small sample of the above referenced polyacrylic acid was dispersed in acetone and similarly scanned. Finally, a small sample of the LUTROL F68 hydroxyl polymer was dissolved in acetone and similarly scanned.

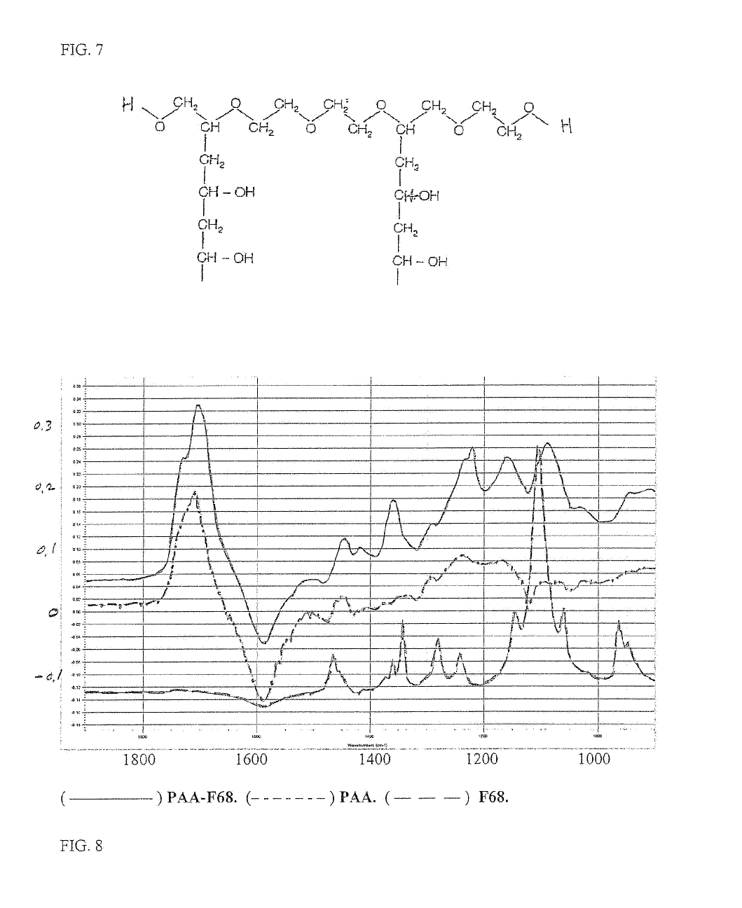

The results of this series of scans produced the infrared signatures are shown in FIG. 8, showing absorbance versus wave number. The solid line curve ( [symbol] ) located upper most portion of the plot represents the signature of the ester complex (i.e., the PAA-F68 ester), the broken line curve with short dashes ( [symbol] ) located in the middle of the plot represents the signature of the polyacrylic acid (PAA), and the bottom curve of broken line with long dashes ( [symbol] ) represents the triblock polymer (F68). The peak absorbance of the carbonyl group in the polyacrylic acid was at 1,710 cm⁻¹. As expected, no carbonyl was detected in the triblock polymer. The peak absorbance of the carbonyl group in the mixture of the polymers after treatment in vacuum and with heat was detected at 1,703 cm⁻¹. This downward shift in the wave number is consistent with the formation of the ester cross-link as the replacement of the hydrogen of the carboxyl group with carbon of the ester group reduces the vibration frequency of the carbonyl oxygen.

A small sample of the PAA:F68 ester polymer was transferred to de-ionized water. The resulting sample swelled in water and formed an elastic hydrogel but did not dissolve. A sample of the PAA was placed in water and it dissolved. Likewise, as sample of LUTROL F68 was placed in water and it dissolved. These three observations provide further evidence for the formation of the ester cross-link. The reaction polymer swelled but did not dissolve because the cross-links prevented the polymer from dissociating into solution.

### Example 6

An ester copolymer of the present invention was formed between the acid polymer polyacrylic acid and a hydroxyl polymer containing an ethylene oxide:propylene oxide:ethylene oxide triblock copolymer. First, 1.2270 grams polyacrylic acid was dissolved in 3.7447 grams water for a concentration of 0.33 g/L. The polyacrylic acid (PAA) was commercially available as CARBOPOL 980 manufactured by Noveon, Incorporated, Cleveland, Ohio. Then, 4.3731 g of the PAA solution was mixed with stirring with 0.6284 grams of LUTROL F127. The triblock copolymer is commercially available as LUTROL® F127 manufactured by the BASF Corporation, Mount Olive, N.J. The mixture was transferred to a vacuum oven and cured at 50° C. in vacuum for 11 days to react and produce the ester polymer of the present invention.

A sample of the resulting ester polymer was placed in water. The sample swelled to an elastic hydrogel but did not dissolve. The unreacted polyacrylic acid and LUTROL F127 each dissolved in water. These observations are consistent with the formation of the ester cross-link between the PAA and the LUTROL F 127. The individual polymers of the reaction were water soluble before the reaction while the ester cross-links after the reaction prevented the polymers from dissolving.

### Example 7

An ester polymer of the present invention was formed between a solid polymer and a liquid polymer without an aqueous solution step and without a pre-drying step. First, 1.4089 grams of polyacrylic acid was mixed with 4.3897 grams of liquid polyethylene glycol (PEG). The polyethylene glycol is commercially available from the Dow Chemical Company, Danbury, Conn., as CARBOWAX® 200. This polymer has an average molecular weight of 200 grams per mole, is a liquid at room temperature, and is a linear polymer with terminal hydroxyl groups at each end of the polymer. The mixture was transferred to a vacuum oven and cured at 50° C. in vacuum for 11 days. A sample of the resulting reacted ester polymer was placed in water which sample swelled but did not dissolve. The polyacrylic acid prior the reaction dissolved in water. Likewise, the PEG prior to the reaction dissolved in water. These observations are consistent with the conclusion that the formation of the ester cross-links allows the reacted polymers to swell but prevents them from dissolving.

### Example 8

An ester polymer of the present invention was formed between a solid polymer and a liquid polymer without an aqueous solution step and without a pre-drying step. First, 1. 1840 grams of polyacrylic acid was mixed with 4.5263 grams of liquid polyethylene glycol (PEG). The polyethylene glycol is commercially available from the Dow Chemical Company, Danbury, Conn., as CARBOWAX® 300. This polymer has an average molecular weight of 300 grams per mole, is a liquid at room temperature, and is a linear polymer with terminal hydroxyl groups at each end of the polymer. The mixture was transferred to a vacuum oven and cured at 50° C. in vacuum for 11 days. A sample of the resulting reacted ester polymer was placed in water which sample swelled but did not dissolve. The polyacrylic acid prior the reaction dissolved in water. Likewise, the PEG prior to the reaction dissolved in water. These observations are consistent with the formation of the ester cross-link that allows the reacted polymers to swell but prevents them from dissolving.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one skilled in the art without departing from the scope of the present invention. All patent and application document references cited in the present disclosure are hereby incorporated by reference in their entireties herein.

What is claimed is:

1. An iontophoretic agent delivery device comprising a pair of electrode assemblies, a least one of said electrode assemblies having a donor electrode and a reservoir for containing a cationic drug to be iontophoretically delivered, said reservoir being applicable in drug transmitting relation with a body surface for iontophoretic delivery, the reservoir having a liquid imbibing polymer with carboxyl groups available for noncovalently associating with the cationic drug.

2. The device of claim 1 wherein the liquid imbibing polymer is dry and associates with the cationic drug before imbibing liquid and is an ester including esterified carboxyl groups and nonesterified carboxyl groups.

3. The device of claim 2 wherein the reservoir prior to hydration being swellable by imbibing liquid, and the liquid imbibing polymer is an ester between an acid polymer and an hydroxyalkyl polymer.

4. The device of claim 2 wherein the liquid imbibing polymer is an ester between an acid polymer and an hydroxyl polymer, the acid polymer being selected from the group consisting of polyacrylic acid, polymethacrylic acid, polyethylacrylic acid, ethyl acrylate/methacrylic acid copolymers, cellulose acetate phthalate, hydroxypropyl methylcellulose acetate succinate, hydroxypropyl methylcellulose phthalate, polyvinyl acetate phthalate, cellulose acetate trimellitate, alginic acid, pectic acid, gelatin, casein, arachin, glycinin, and zein; and the hydroxyl polymer being selected from the group consisting of hydroxyethyl cellulose, hydroxypropyl cellulose, hydroxypropyl methyl cellulose, starch, maltodextrin, chitosan, polyvinyl alcohol, polyethylene glycol, ethylene oxide, ethylene oxide:propylene oxide:ethylene oxide triblock copolymer, and polyvinyl alcohol-polyethylene glycol graft copolymer.

5. The device of claim 3 wherein the acid polymer is polyacrylic acid and the hydroxyalkyl polymer is hydroxyalkyl cellulose.

6. The device of claim 3 wherein the acid polymer is one of polyacrylic acid polymer and polymethacrylic acid polymer and the hydroxyalkyl polymer is hydroxyethyl cellulose.

7. The device of claim 3 wherein the acid polymer contains polyacrylic acid and the hydroxyalkyl polymer contains primary hydroxyl groups connected to a hydrocarbon chain that is connected via ether linkage to another group in the hydroxyalkyl polymer.

8. The device of claim 3 comprising a cationic drug that is recoverable at 90% or less from an aqueous solution over a period of 1 week at room temperature.

9. The device of claim 3 wherein the hydroxyalkyl polymer is a polymer of at least one of ethylene glycol, ethylene oxide, and propylene oxide.

10. The device of claim 3 wherein the hydroxyalkyl polymer is hydroxyalkyl polysaccharide derivative.

11. The device of claim 3 wherein the acid polymer is either acrylic acid homopolymer or copolymer of acrylic acid and alkyl acrylate, the hydroxyalkyl polymer is a hydroxyalkyl polysaccharide derivative and the liquid imbibing polymer formed therefrom when dry is hydratable by imbibing an aqueous solution up to 75 wt %.

12. A method of forming an iontophretic drug delivery device, comprising: preparing a hydratable reservoir by drying a wet gel that contains a cationic drug such that the hydratable reservoir contains a liquid-imbibing polymer having nonesterified carboxyl groups for noncovalently associating with the cationic drug, the hydratable reservoir can be hydrated by infusing a liquid thereto to form a gel for electrotransport.

13. The method of claim 12 comprising providing the liquid to the hydratable reservoir and wherein the liquid imbibing polymer is formed by esterification to result in esterified carboxyl groups and nonesterified carboxyl groups in the polymer.

14. The method of claim 13 comprising reacting an acid polymer that is one of polyacrylic acid polymer and polymethacrylic acid polymer with an hydroxyalkyl polymer in the esterification.

15. The method of claim 13 wherein the liquid imbibing polymer is formed by esterification between an acid polymer and an hydroxylalkyl polymer, the acid polymer being selected from the group consisting of polyacrylic acid, polymethacrylic acid, polyethylacrylic acid, ethyl acrylate/methacrylic acid copolymers, cellulose acetate phthalate, hydroxypropyl methylcellulose acetate succinate, hydroxypropyl methylcellulose phthalate, polyvinyl acetate phthalate, cellulose acetate trimellitate, alginic acid, pectic acid, gelatin, casein, arachin, glycinin, and zein; and the hydroxylalkyl polymer being selected from the group consisting of hydroxyethyl cellulose, hydroxypropyl cellulose, hydroxypropyl methyl cellulose, starch, maltodextrin, chitosan, polyvinyl alcohol, polyethylene glycol, ethylene oxide, ethylene oxide:propylene oxide:ethylene oxide triblock copolymer, and polyvinyl alcohol-polyethylene glycol graft copolymer.

16. The method of claim 14 wherein the liquid imbibing polymer is formed by esterification between a polyacrylic acid and a hydroxyalkyl cellulose.

17. The method of claim 14 wherein the liquid imbibing polymer is formed by esterification between polyacrylic acid and hydroxyethyl cellulose.

18. The method of claim 14 wherein the liquid imbibing polymer is formed by esterification between a polyacrylic acid and a hydroxyalkyl polymer containing primary hydroxyl groups connected to a hydrocarbon chain which is connected via ether linkage to another group in the hydroxyalkyl polymer.

19. The method of claim 14 wherein the liquid imbibing polymer is formed by esterification between a polyacrylic acid and an hydroxyl polymer that is a polymer of at least one of ethylene glycol, ethylene oxide, and propylene oxide.

20. The method of claim 14 wherein the acid polymer is either acrylic acid homopolymer or copolymer of acrylic acid and alkyl acrylate, the hydroxyalkyl polymer is a hydroxyalkyl polysaccharide derivative and the liquid imbibing polymer formed therefrom when dry is hydratable by imbibing an aqueous solution up to 75 wt %.

21. The method of claim 13 comprising contacting a hydratable liquid-imbibing polymer with a cationic drug solution to form the wet gel and then dehydrating the wet gel to form the hydratable reservoir and comprising allowing the hydratable reservoir to imbibe 15 vol % to 50 vol % liquid.

22. A manufacture for use for iontophoretic drug delivery, comprising a pair of electrode assemblies, a least one of said electrode assemblies having a donor electrode and a reservoir for containing a cationic drug to be iontophoretically delivered, said reservoir being hydratable to be placed in drug transmitting relation with a body surface for iontophoretic delivery, the reservoir having a liquid imbibing polymer with carboxyl groups nonesterified for noncovalently associating with the cationic drug.

23. The manufacture of claim 22 wherein the reservoir is dry and the liquid imbibing polymer is an ester between an acid polymer and an hydroxylalkyl polymer, the acid polymer being selected from the group consisting of polyacrylic acid, polymethacrylic acid, polyethylacrylic acid, ethyl acrylate/methacrylic acid copolymers, cellulose acetate phthalate, hydroxypropyl methylcellulose acetate succinate, hydroxypropyl methylcellulose phthalate, polyvinyl acetate phthalate, cellulose acetate trimellitate, alginic acid, pectic acid, gelatin, casein, arachin, glycinin, and zein; and the hydroxylalkyl polymer being selected from the group consisting of hydroxyethyl cellulose, hydroxypropyl cellulose, hydroxypropyl methyl cellulose, starch, maltodextrin, chitosan, polyvinyl alcohol, polyethylene glycol, ethylene oxide, ethylene oxide:propylene oxide:ethylene oxide triblock copolymer, and polyvinyl alcohol-polyethylene glycol graft copolymer.

24. The device of claim 23 wherein the acid polymer is polyacrylic acid and the hydroxyalkyl polymer is hydroxyalkyl cellulose.

25. An iontophoretic agent delivery device comprising a pair of electrode assemblies, a least one of said electrode assemblies having a donor electrode and a reservoir for containing a cationic drug to be iontophoretically delivered, said reservoir being applicable in drug transmitting relation with a body surface for iontophoretic delivery, the reservoir having a liquid imbibing polymer with carboxyl groups available for noncovalently associating with the cationic drug, the liquid imbibing polymer being an ester polymer having an acid polymer monomeric component and an hydroxyalkyl polymer monomeric component, the acid polymer monomeric component being one of polyacrylic acid polymer and polymethacrylic acid polymer and the hydroxyalkyl polymer monomeric component is hydroxyalkyl cellulose.
